(19) United States
(12) Patent Application Publication
(10) Pub. No.: US 2022/0268771 A1
(43) Pub. Date: Aug. 25, 2022

(54) COMPOSITIONS AND METHODS FOR LYME DISEASE

(71) Applicant: The Johns Hopkins University, Baltimore, MD (US)

(72) Inventor: Ying Zhang, Ellicott City, MD (US)

(21) Appl. No.: 17/622,037

(22) PCT Filed: Jun. 26, 2020
(86) PCT No.: PCT/US2020/039884

§ 371 Date: Dec. 22, 2021

Related U.S. Application Data
(60) Provisional application No. 62/867,194, filed on Jun. 26, 2019.

(51) Int. Cl. G01N 33/569; G01N 33/49

(52) U.S. Cl. CPC G01N 33/56911; G01N 33/4915; G01N 2333/20; G01N 2469/20

(57) ABSTRACT

Provided herein are, inter alia, methods and kits for diagnosing post-treatment Lyme disease syndrome (PTLDS) or Lyme disease in a subject.

### PRIORITY CLAIM

This application claims benefits of priority to U.S. Provisional Application No. 62/867,194 filed Jun. 26, 2019, the entire contents of which are incorporated herein by reference.

### BACKGROUND

New compositions and methods for more sensitive diagnosis of Lyme disease are needed.

### BRIEF SUMMARY

Provided herein are, inter alia, methods, compositions and kits for diagnosing and treating Lyme disease and post-treatment Lyme disease syndrome (PTLDS).

In certain preferred embodiments, provided herein are methods for diagnosing Lyme disease in a subject, the method comprising obtaining a sample from the subject, assaying the level of antibodies to antigens from unique microcolonies of Borrelia burgdorferi (e.g., the antigens are derived from microcolony persister antigens (MPA)), and diagnosing the subject with Lyme disease and infections due to related Borrelia species (described herein) when detection of the antibodies in the sample is observed.

In additional preferred embodiments, methods are provided for diagnosing Lyme disease in a subject, the method comprising obtaining a sample from the subject, assaying the sample for level of antigens from unique microcolonies of Borrelia burgdorferi (e.g., the antigens are microcolony persister form antigens (MPA)), and diagnosing the subject with Lyme disease and infections due to related Borrelia species (described herein) when detection of the antibodies to MPA or the MPA in the sample is observed.

Methods for preparation of microcolony persister antigens (MPA) of Borrelia burgdorferi for improved serodiagnosis of Lyme disease, including early Lyme disease and PTLDS are also provided herein.

The methods for preparation of microcolony persister antigens of Borrelia burgdorferi, include Borrelia burgdorferi sensu lato species, to include B. afzelii, B. garinii, B. bavariensis, B. garinii, B. japonica, B. lusitaniae, B. sinica, B. spielmanii, B. tanukii, B. turdi, B. valaisiana, and B. yangtze, B. bissettii, and B. carolinensis, B. americana, B. andersonii, B. californiensis, B. carolinensis, and B. kurtenbachii), as well as related borrelia species such as B. valaisiana and B. miyamotoi.

In embodiments, the subject is diagnosed with the Lyme disease if the level of the antibodies to antigens comprising MPA is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in said test sample compared to a normal control.

In embodiments, the method further comprises use of detecting antibody to C6 peptide comprising MKKDDQIAAAIALRGMAKDGKFAVKDGE (SEQ ID NO: 1) in combination with the antigens comprising MPA for improved diagnosis of Lyme disease and or PTLDS.

The methods described herein further comprises detecting antibody to C6 peptide comprising MKKDDQIAAAIALRGMAKDGKFAVKDGE (SEQ ID NO: 1) in combination with detecting the antibody to MPA.

In embodiments, the Lyme disease comprises early Lyme disease. For example, early Lyme disease refers to the period within the first 4-6 weeks after the tick bite, and is not early disseminated disease.

In embodiments, the sample comprises a bodily fluid from the subject, e.g., the sample includes bodily fluid comprising whole blood, a component of whole blood, plasma, serum, urine, cerebrospinal fluid, or synovial fluid, which contain antibodies to MPAs.

In embodiments, the method comprises assaying the level of antibodies to MPAs. In further embodiments, the assaying comprises an enzyme-linked immunosorbent assay (ELISA), an antigen capture assay, lateral flow assay, quantum dot, flow cytometry, immunoblot, a Western blot, a mass spectrometry assay, immunoprecipitation, immunodiffusion, immunocytochemistry, radioimmunoassay, or any combination thereof.

In aspects, also provided herein are methods of detecting post-treatment Lyme disease syndrome (PTLDS) or persistent (chronic) Lyme disease in a subject, comprising obtaining a sample from the subject, detecting the presence of antibodies to antigens from Borrelia burgdorferi, or detecting antigens from Borrelia burgdorferi, wherein the antigens comprise microcolony persister form antigens (MPAs), and wherein detection of the MPA in the sample is indicative of PTLDS or Lyme disease or persistent (chronic) Lyme disease.

In other aspects, provided herein are methods for increasing the sensitivity of serodiagnosis for early Lyme disease and post-treatment Lyme disease syndrome in a subject, comprising obtaining a sample from the subject, detecting the presence of antibodies to antigens from Borrelia burgdorferi, or detecting antigens from Borrelia burgdorferi, wherein the antigens comprise microcolony persister form antigens (MPA), and wherein detection of both the MPA in the sample is indicative of PTLDS or Lyme disease and increases the sensitivity of serodiagnosis. In embodiments, the method further comprises detecting C6 peptide comprising MKKDDQIAAAIALRGMAKDGKFAVKDGE (SEQ ID NO: 1), and wherein detection of both the MPA and C6 in combination in the sample is indicative of PTLDS or Lyme disease and increases the sensitivity of serodiagnosis.

In aspects, provided here in is a kit comprising at least two agents selected from the group consisting of (i) microcolony persister form antigens (MPAs); and (ii) a C6 peptide, and instructions for diagnosing a Lyme disease or post-treatment Lyme disease syndrome (PTLDS), for identifying whether a subject is at risk of developing the Lyme disease or PTLDS, for determining the prognosis of the Lyme disease or PTLDS, for determining the progression of Lyme disease or PTLDS, for assessing the efficacy of a treatment for the Lyme disease or PTLDS, and the presence of antibodies to MPA as a biomarker for PTLDS, the reduced level of antibodies to the MPA as an indicator of treatment efficacy, and/or for adjusting the dose of a compound during the treatment of Lyme disease or PTLDS.

In other aspects, a diagnostic system is provided that comprises an assortment, collection, or compilation of test results data representing the level of antibodies to antigens from Borrelia burgdorferi, wherein the antigens comprise microcolony persister form antigens (MPAs) or C6 peptide in a plurality of test samples; a means for computing an index value using said level, wherein the index value comprises a diagnostic, prognostic, progression, or treatment response; and a means for reporting the index value.

Diagnostic systems are also provided that comprises an assortment, collection, or compilation of test results representing the level antigens from Borrelia burgdorferi, wherein the antigens comprise microcolony persister form antigens (MPA) or C6 peptide in a plurality of test samples; a means for computing an index value using said level, wherein the index value comprises a diagnostic, prognostic, progression, or treatment response; and a means for reporting the index value.

In further aspects, methods are provided for determining the efficacy of a therapeutic regimen in a subject (e.g., a mammal, including a human subject) having Lyme disease. For example, the method comprises, detecting the presence of antibodies to antigens from Borrelia burgdorferi, wherein the antigens comprise microcolony persister form antigens (MPA), in the subject prior to the initiation of treatment, detecting the presence of antibodies to antigens from Borrelia burgdorferi, wherein the antigens comprise microcolony persister antigens (MPAs) in the subject after initiation of the therapeutic regimen, and a decrease in the antibodies to antigens from Borrelia burgdorferi is a positive indicator of the efficacy of the therapeutic regimen.

The methods also may comprises detecting the presence of antigens from Borrelia burgdorferi, wherein the antigens comprise microcolony persister form antigens (MPA), in the subject prior to the initiation of treatment, detecting the presence of antigens from Borrelia burgdorferi, wherein the antigens comprise microcolony persister form antigens (MPA) in the subject after initiation of the therapeutic regimen, and a decrease in the antigens from Borrelia burgdorferi is a positive indicator of the efficacy of the therapeutic regimen.

In embodiments, a decrease in the antibodies of at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in said test sample compared to a normal control is a positive indicator of the efficacy of the therapeutic regimen.

In embodiments, the efficacy of the therapeutic regimen is determined once a week, or once every two weeks, or once every 3 weeks or once every 4 weeks. In some embodiments, the efficacy of the therapeutic regimen is determined for 1 to 4 months, from 2 to 6 months, from 2 to 8 months, from 2 to 10 months, or from 2 to 12 months, 2 to 16 months, or longer (e.g., 1, 2, 3, 4, 5, 6, 7, 8, 9, 10, 11, 12, 36, 48, or more months).

In embodiments, the therapeutic regimen comprises administering an antibiotic to the subject, for example, the antibiotic is doxycycline, or amoxicillin, or cefuroxime, or ceftriaxone.

Other aspects of the invention are disclosed infra.

### DESCRIPTION OF THE DRAWINGS

FIGS. 1A-1C show images of the isolation of variant forms of B. burgdorferi during its natural growth cycle from log phase to stationary phase. A 4-day old B. burgdorferi culture consisting of exclusively growing spirochetes is considered as log phase (FIG. 1A); Planktonic (spirochete and round body) form (FIG. 1B) and aggregated biofilm-like microcolony form (FIG. 1C) were isolated from the 10-day old stationary phase culture. The different forms were stained by SYBR Green I assay and observed by microscopy as described previously (Feng et al., 2014). Scale bar (in Panel A)=100 μm.

FIGS. 2A and 2B show data of antibody response to antigens from three different forms of Borrelia with Lyme disease patient sera. Whole cell lysates prepared from three different forms of Borrelia were separated on SDS-PAGE gel and stained by Coomassie Blue (FIG. 2A). Microtiter wells were coated with 100 ng of lysates prepared from three different forms of Borrelia. Immunoglobulin G/M responses to Borrelia spirochetes from Log phase (LOG.Bb) or Planktonic forms (SP.Bb) or biofilm-like microcolonies (MC.Bb) (FIG. 2B). The antibody response (expressed as OD readings on the Y-axis) of individual serum samples from control donors (control, black circles, N=25) and patients with Lyme disease (EM rash or PTLDS, N=65) to different antigens was measured by ELISA. Positive counts from patient samples are labeled as red blocks and negative counts from patient samples are labeled black blocks. The cut-off OD value was calculated for the antigens from healthy controls. For all antigens, borderline responses (mean+2 SDs) were included in positive results.

FIGS. 3A and 3B are graphs showing antibody response to antigens from Borrelia spirochetes and biofilm-like microcolonies with Lyme disease patient sera. Microtiter wells were coated with 100 ng of lysates prepared from different forms of Borrelia. Immunoglobulin M (IgM) and Immunoglobulin G (IgG) responses to Borrelia spirochetes (FIG. 3A) and biofilm-like microcolonies (FIG. 3B). The antibody response (expressed as OD readings on the Y-axis) of individual serum samples from healthy controls (control, black circles, N=25) and patients with Lyme disease (EM rash or PTLDS, N=65) to different antigens was measured by ELISA. Positive counts were labeled in red and the cut-off OD value was calculated for the antigens from healthy controls. For all antigens, borderline responses (mean+2 SDs) were included in positive results.

FIG. 4 is a graph showing the antibody response to Borrelia spirochetes and biofilm-like microcolonies increases reaction of C6 peptide to Lyme disease patient sera. Antibody responses from 65 patient samples from Columbia to different antigen preps or C6 alone or in combination were analyzed by ELISA. Overall positive IgM and IgG reactions to C6 peptide, Spirochetes, MPAs, C6 peptide plus Spirochetes and C6 peptide plus MPAs. ***p<0.001, **p<0.01, *p<0.05 by a Chi-square test.

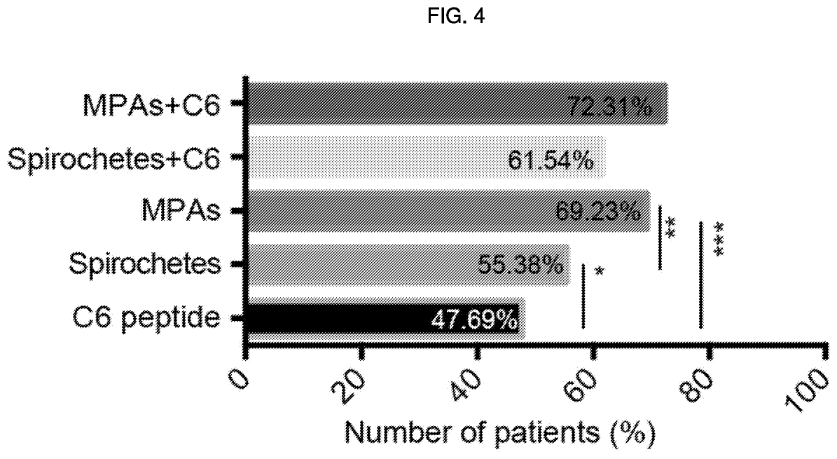

FIGS. 5A and 5B are graphs showing that microcolony persister antigens increased IgM and IgG response to well-characterized early Lyme and PTLDS serum samples in the SLICE cohorts. Microtiter wells were coated with 100 ng of different forms of Borrelia lysate. IgM and IgG responses by patients to LOG.Bb (FIG. 5A) and MC.Bb (FIG. 5B) were measured by ELISA as described herein. The antibody response of individual serum samples from control donors (control, black circles, total 20 samples), patients with early Lyme disease (EM, erythema migrans, black square, total 60 samples), patients with late Lyme disease (PTLDS, post-treatment Lyme disease syndrome, black triangle, total 60 samples) were measured by ELISA. Positive counts were labeled in red and the cutoff OD value was calculated for the antigens from control donors.

FIGS. 6A and 6B are graphs showing that the combination of MPAs and C6 peptide increased the sensitivity of diagnosis for both early Lyme and PTLDS using the well-characterized serum samples from SLICE studies. (FIG. 6A) IgM (left panel) response and IgG (right panel) response by early Lyme or PTLDS patient sera to C6 peptide. FIG. 6B is a graph depicting the overall positive IgM and IgG responses to C6 peptide, MPAs, and C6 peptide plus MPAs in early Lyme and PTLDS serum samples. ***p<0.001, **p<0.01, *p<0.05 by a Chi-square test.

FIG. 7 is an image of a blot, wherein Lane 1 is the log phase Borrelia burgdorferi cell lysate; Lane 2. Microcolony cell lysate; Lane 3. Stationary phase cell lysate from B. burgdorferi N40 strain. Patient A (700008) and patient B (600011). Unique band from microcolony cell lysate as antigen are labeled with *. Primary antibody: patient sera (1:100 dilution). Secondary antibody: Goat anti-human IgG/A/M (Invitrogen A18847) (1:5000 dilution).

### DETAILED DESCRIPTION

Provided herein are, inter alia, methods, compositions and kits for diagnosing Lyme disease and post-treatment Lyme disease syndrome (PTLDS), or monitoring the treatment thereof.

Lyme disease, caused by Borrelia burgdorferi, is the most common vector-borne disease and increasing public health problem in the US and Europe. There are estimated 300,000 cases a year in the US (1). Lyme disease is a multisystemic disease that is initiated by tick bite that carries B. burgdorferi (2). Untreated Lyme disease has three stages, early localized disease with an erythema migrans (EM) rash, early disseminated disease such as Bell's palsy and carditis, and late disseminated disease characterized by arthritis and neurological symptoms (3). Prompt treatment in most cases can prevent the disease from developing into chronic persisting symptoms such as fatigue, musculoskeletal pain, arthritis, and neurological impairment. The current standard treatment with 2-4 week doxycycline antibiotic treatment cures Lyme disease in 80-90% of the patients (4). However, about 10-20% of patients develop persisting symptoms of fatigue, pain, and neurological symptoms despite the standard antibiotic treatment, a condition called post-treatment Lyme disease syndrome (PTLDS) (5). However, proper diagnosis and treatment of Lyme disease in the early stage is imperative to limiting the number of patients who progress to later, more severe disease. Although clinical diagnosis of early Lyme disease relies on identification of EM rash and a history of exposure, a significant portion of patients do not have EM rash or typical EM rash, making diagnosis difficult. Culture and PCR of the skin lesions or blood have a low sensitivity of around 50% and even lower for late stage disease and are therefore not routinely used for diagnosis of Lyme disease. The Centers for Disease Control and Prevention (CDC) recommends a two-tiered serologic test to detect the patient's antibody response to B. burgdorferi antigens (1, 6, 7). The first-tier consists of an enzyme-linked immunoassay (EIA) with whole cell lysates of B. burgdorferi, followed by a second-tier Western blot or a C6 peptide test (2). While the sensitivity of these tests is quite high for disseminated Lyme disease (>82%) (7), the sensitivity remains low for diagnosis of early stage Lyme disease with EM rash (30-40%) (3) and also for post-treatment Lyme disease syndrome (PTLDS) (50-60%) (3-7).

One disadvantage of the 2-tiered testing approach is that Western blot is too tedious. Recent studies indicate that C6 peptide, derived from VlsE protein when used as a stand-alone test is more sensitive than the current 2-tiered test for patients with early Lyme disease (64% vs. 48%) with comparable specificity (98.4% vs 99.5%) (8). Interestingly, when C6 test is added together with ELISA in a two-tiered ELISA format which is much simpler than ELISA plus Western blot and removes the complexity of the Western Blot, an increase in sensitivity in detecting early disease up to slightly over 50% sensitivity, with very good performance in Stage 2 and in Stage 3 illness 100% of the samples were observed (6-8). This indicates that it is possible to add other antigens together with C6 to improve the sensitivity of the current Lyme diagnosis. However, it remains a significant challenge to diagnose early Lyme disease as well as late stage PTLDS patients with the current two-tier test or even the new improved C6 plus whole cell lysate one tier test.

It has been demonstrated that stationary phase B. burgdorferi cultures develop multiple morphological forms including spirochetes, round bodies, aggregated biofilm-like microcolonies that are different from log phase culture which primarily consists of spirochetal form (9). Importantly, these variant forms have different biological properties as round body and microcolony forms have been shown to be dormant persisters that are more persistent or tolerant to antibiotics than the spirochetal form (9, 10). In addition, the morphological variant forms have different ability to induce host cytokine response (11) and also have different ability to cause disease in a recent mouse study (10). However, the utility of the antigens derived from the variant forms that develop in old stationary phase cultures for serodiagnosis of Lyme disease has not been evaluated.

As described herein, the inclusion of antigens derived from persister forms of Borrelia bacteria (12, 13) improved the diagnosis of Lyme disease according to the Yin-Yang model so as to include antigens from both growing bacteria and non-growing persister bacteria (12, 14). Here, the antigens prepared from different forms of B. burgdorferi (planktonic round body form and microcolony form from stationary phase culture, and spirochete from log phase culture, and water-induced round bodies) for serodiagnosis of Lyme disease were evaluated in comparison with the current two-tier and C6 single tier tests.

Interestingly, it was found that only the antigens derived from the microcolony persister form, but not those from round bodies or spirochete form (either from log or stationary phase), provide significantly better sensitivity than the current Lyme tests in terms of diagnosing Lyme disease, especially PTLDS patients. These findings not only improve the serodiagnosis of Lyme disease but also have implications for understanding pathogenesis of PTLDS.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “disease” refers to any deviation from the normal health of a mammal and includes a state when disease symptoms are present, as well as conditions in which a deviation (e.g., Lyme disease, in particular early stage Lyme disease, and post-treatment Lyme disease syndrome (PTLDS)) has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with Lyme disease, in particular early stage Lyme disease, and post-treatment Lyme disease syndrome (PTLDS), and compared to samples from known conditions, e.g., a subject (or subjects) that does not have Lyme disease, in particular early stage Lyme disease, and post-treatment Lyme disease syndrome (PTLDS) (a negative or normal control), or a subject (or subjects) who does have Lyme disease, in particular early stage Lyme disease, and post-treatment Lyme disease syndrome (PTLDS)) (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have Lyme disease, in particular early stage Lyme disease, and post-treatment Lyme disease syndrome (PTLDS) in a healthy or general population. The amount of a compound can be measured in a test sample and compared to the “normal control” level, utilizing techniques such as reference limits, discrimination limits, or risk defining thresholds to define cutoff points and abnormal values (e.g., for Lyme disease, in particular early stage Lyme disease, and post-treatment Lyme disease syndrome (PTLDS) or a symptom thereof). The normal control level means the level of one or more compounds or combined compounds typically found in a subject known not suffering from Lyme disease, in particular early stage Lyme disease, and post-treatment Lyme disease syndrome (PTLDS). Such normal control levels and cutoff points may vary based on whether a compound is used alone or in a formula combining with other compounds into an index. Alternatively, the normal control level can be a database of compounds patterns from previously tested subjects who did not develop Lyme disease, in particular early stage Lyme disease, and post-treatment Lyme disease syndrome (PTLDS) or a particular symptom thereof (e.g., in the event the Lyme disease, in particular early stage Lyme disease, and post-treatment Lyme disease syndrome (PTLDS) develops or a subject already having Lyme disease, in particular early stage Lyme disease, and post-treatment Lyme disease syndrome (PTLDS) is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA or antibody level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA or antibody level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1-2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. 1307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982))

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “epitope” as used herein, refers to a portion of an antigen that is specifically recognized by an antibody.

In embodiments, the antibodies to MPA (e.g., MPA to detect patient antibodies to MPA) described herein may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice, or human), e.g., the antibody comprises a monoclonal antibody.

An “isolated antibody,” as used herein, is intended to refer to an antibody that is substantially free of other antibodies having different antigenic specificities (e.g., antibodies to MPA (e.g., MPA to detect patient antibodies to MPA)). Moreover, an isolated antibody may be substantially free of other cellular material and/or chemicals.

The antibody of the present invention may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice).

The terms “monoclonal antibody” or “monoclonal antibody composition” as used herein refer to a preparation of antibody molecules of single molecular composition. A monoclonal antibody composition displays a single binding specificity and affinity for a particular epitope.

An “antibody fragment” comprises a portion of an intact antibody, preferably the antigen binding and/or the variable region of the intact antibody. Non-limiting examples of antibody fragments include Fab, Fab*, F(ab′)₂ and Fv fragments; diabodies; linear antibodies; single-chain antibody molecules and multispecific antibodies formed from antibody fragments.

The invention may further comprise a humanized antibody, wherein the antibody is from a non-human species, whose protein sequence has been modified to increase their similarity to antibody variants produced naturally in humans. Generally, a humanized antibody has one or more amino acid residues introduced into it from a source which is non-human. These non-human amino acid residues are referred to herein as “import” residues, which are typically taken from an “import” antibody domain, particularly a variable domain.

The term “recombinant human antibody,” as used herein, includes all human antibodies that are prepared, expressed, created or isolated by recombinant means, such as (a) antibodies isolated from animals (e.g., sheep, rabbits, goats, or mice) that are transgenic or transchromosomal for human immunoglobulin genes, (b) antibodies isolated from a host cell transformed to express the human antibody, (c) antibodies isolated from a recombinant, combinatorial human antibody library, and (d) antibodies prepared, expressed, created or isolated by any other means that involve splicing of human immunoglobulin gene sequences to other DNA sequences.

An “antibody fragment” comprises a portion of an intact antibody, preferably the antigen binding and/or the variable region of the intact antibody. Examples of antibody fragments include Fab, Fab*, F(ab′)₂ and Fv fragments; diabodies; linear antibodies; single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “antigen” as used herein, refers to a protein or polypeptide capable of generating an immune response in the form of an antibody. An antigen may comprise one or more epitopes that bind specific antibodies. “Antigen” also refers to a molecule that provokes an immune response. This immune response may involve either antibody production, or the activation of specific immunologically-competent cells, or both. For example, any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein.

The term “Borrelia species” as used herein, refers to any Borrelia species known to cause Lyme disease or Lyme-like illness. Non-limiting examples include Borrelia afzelii, Borrelia burgdorferi, Borrelia garinii, Borrelia miyamotoi and Borrelia valaisiana.

The term “detection moiety” as used herein, refers to a binding partner attached to a label that is detectable and/or capable of producing a detectable signal. The label can be attached, directly or indirectly, to various binding partners. An example of a detection moiety is a detector antibody that may be an anti-human antibody that is capable of binding to the antibody portion of the antibody-antigen complex formed in the methods disclosed herein. Commercially available anti-human antibodies may be suitable for use with the methods herein. Other detection. antibodies for other species may be used.

The term “solid support” as used herein, refers to any material that is insoluble and/or has structural rigidity and resistance to changes of shape or volume, and to which an antigen can be immobilized or bound.

The term “persister form” as used herein, refers to any dormant, metabolically quiescent cells tolerant to stresses and drugs or antibiotics including microbial cells (bacteria, fungi, parasites) which has a different protein expression than an actively growing cell.

### Lyme Disease

Lyme disease, also known as Lyme borreliosis, is an infectious disease caused by a bacterium named Borrelia spread by ticks. The most common sign of infection is an expanding area of redness on the skin, known as erythema migrans, that appears at the site of the tick bite about a week after it occurred. The rash is typically neither itchy nor painful. Approximately 70-80% of infected people develop a rash. Other early symptoms may include fever, headache and tiredness. If untreated, symptoms may include loss of the ability to move one or both sides of the face, joint pains, severe headaches with neck stiffness, or heart palpitations, among others. Months to years later, repeated episodes of joint pain and swelling may occur. Occasionally, people develop shooting pains or tingling in their arms and legs. Despite appropriate treatment, about 10 to 20% of people develop joint pains, memory problems, and tiredness for at least six months.

Early Localized Infection

Early localized infection can occur when the infection has not yet spread throughout the body. Only the site where the infection has first come into contact with the skin is affected. The initial sign of about 80% of Lyme infections is an Erythema migrans (EM) rash at the site of a tick bite, often near skin folds, such as the armpit, groin, or back of knee, on the trunk, under clothing straps, or in children's hair, ear, or neck. Most people who get infected do not remember seeing a tick or the bite. The rash appears typically one or two weeks (range 3-32 days) after the bite and expands 2-3 cm per day up to a diameter of 5-70 cm (median 16 cm). The rash is usually circular or oval, red or bluish, and may have an elevated or darker center.

Early Disseminated Infection

Within days to weeks after the onset of local infection, the Borrelia bacteria may spread through the lymphatic system or bloodstream. In 10-20% of untreated cases, EM rashes develop at sites across the body that bear no relation to the original tick bite. Transient muscle pains and joint pains are also common.

In about 10-15% of untreated people, Lyme causes neurological problems known as neuroborreliosis. Early neuroborreliosis typically appears 4-6 weeks (range 1-12 weeks) after the tick bite and involves some combination of lymphocytic meningitis, cranial neuritis, radiculopathy and/or mononeuritis multiplex. Lymphocytic meningitis causes characteristic changes in the cerebrospinal fluid (CSF) and may be accompanied for several weeks by variable headache and, less commonly, usually mild meningitis signs such as inability to flex the neck fully and intolerance to bright lights, but typically no or only very low fever. Lyme radiculopathy is an inflammation of spinal nerve roots that often causes pain and less often weakness, numbness, or altered sensation in the areas of the body served by nerves connected to the affected roots, e.g. limb(s) or part(s) of trunk. Mononeuritis multiplex is an inflammation causing similar symptoms in one or more unrelated peripheral nerves.

Late Disseminated Infection

After several months, untreated or inadequately treated people may go on to develop chronic symptoms that affect many parts of the body, including the joints, nerves, brain, eyes, and heart. Lyme arthritis occurs in up to 60% of untreated people, typically starting about six months after infection. It usually affects only one or a few joints, often a knee or possibly the hip, other large joints, or the temporomandibular joint. There is usually large joint effusion and swelling, but only mild or moderate pain. Without treatment, swelling and pain typically resolve over time but periodically return. Baker's cysts may form and rupture. In some cases, joint erosion occurs.

Chronic neurologic symptoms occur in up to 5% of untreated people. A peripheral neuropathy or polyneuropathy may develop, causing abnormal sensations such as numbness, tingling or burning starting at the feet or hands and over time possibly moving up the limbs. A neurologic syndrome called Lyme encephalopathy is associated with subtle memory and cognitive difficulties, insomnia, a general sense of feeling unwell, and changes in personality. Lyme can cause a chronic encephalomyelitis that resembles multiple sclerosis. It may be progressive and can involve cognitive impairment, brain fog, migraines, balance issues, weakness in the legs, awkward gait, facial palsy, bladder problems, vertigo, and back pain. In rare cases, untreated Lyme disease may cause frank psychosis, which has been misdiagnosed as schizophrenia or bipolar disorder.

Late disseminated infection is also characterized as Persistent Lyme disease or PTLDS or chronic Lyme disease.

Pathophysiology

B. burgdorferi can spread throughout the body during the course of the disease, and has been found in the skin, heart, joints, peripheral nervous system, and central nervous system. Many of the signs and symptoms of Lyme disease are a consequence of the immune response to the spirochete in those tissues. B. burgdorferi is injected into the skin by the bite of an infected Ixodes tick. Tick saliva, which accompanies the spirochete into the skin during the feeding process, contains substances that disrupt the immune response at the site of the bite. This provides a protective environment where the spirochete can establish infection. The spirochetes multiply and migrate outward within the dermis. The host inflammatory response to the bacteria in the skin causes the characteristic circular EM lesion. Neutrophils, however, which are necessary to eliminate the spirochetes from the skin, fail to appear in the developing EM lesion. This allows the bacteria to survive and eventually spread throughout the body.

Days to weeks following the tick bite, the spirochetes spread via the bloodstream to joints, heart, nervous system, and distant skin sites, where their presence gives rise to the variety of symptoms of the disseminated disease. The spread of B. burgdorferi is aided by the attachment of the host protease plasmin to the surface of the spirochete. If untreated, the bacteria may persist in the body for months or even years, despite the production of B. burgdorferi antibodies by the immune system. The spirochetes may avoid the immune response by decreasing expression of surface proteins that are targeted by antibodies, antigenic variation of the VlsE surface protein, inactivating key immune components such as complement, and hiding in the extracellular matrix, which may interfere with the function of immune factors.

In the brain, B. burgdorferi may induce astrocytes to undergo astrogliosis (proliferation followed by apoptosis), which may contribute to neurodysfunction. The spirochetes may also induce host cells to secrete quinolinic acid, which stimulates the NMDA receptor on nerve cells, which may account for the fatigue and malaise observed with Lyme encephalopathy. In addition, diffuse white matter pathology during Lyme encephalopathy may disrupt gray matter connections, and could account for deficits in attention, memory, visuospatial ability, complex cognition, and emotional status. White matter disease may have a greater potential for recovery than gray matter disease, perhaps because the neuronal loss is less common. Resolution of MRI white matter hyperintensities after antibiotic treatment has been observed.

Immunological Studies

Exposure to the Borrelia bacterium during Lyme disease possibly causes a long-lived and damaging inflammatory response, a form of pathogen-induced autoimmune disease. The production of this reaction might be due to a form of molecular mimicry, where Borrelia avoids being killed by the immune system by resembling normal parts of the body's tissues.

Diagnosis and Currently Available Treatments

Lyme disease is diagnosed based on symptoms, objective physical findings (such as erythema migrans (EM) rash, facial palsy, or arthritis), history of possible exposure to infected ticks, and possibly laboratory tests. People with symptoms of early Lyme disease should have a total body skin examination for EM rashes and be inquired if there was one in the past 1-2 months. Presence of an EM rash and recent tick exposure (i.e., being outdoors in a likely tick habitat where Lyme is common, within 30 days of the appearance of the rash) are sufficient for Lyme diagnosis; no laboratory confirmation is needed or recommended.

In the absence of an EM rash or history of tick exposure, Lyme diagnosis depends on laboratory confirmation. The bacteria that cause Lyme disease are difficult to observe directly in body tissues and also difficult and too time-consuming to grow in the laboratory. The most widely used tests look instead for presence of antibodies against those bacteria in the blood. A positive antibody test result does not by itself prove active infection, but can confirm an infection that is suspected because of symptoms, objective findings, and history of tick exposure in a person. In some cases, when history, signs, and symptoms are strongly suggestive of early disseminated Lyme disease, empiric treatment may be started and reevaluated as laboratory test results become available.

Laboratory Testing

Tests for antibodies in the blood by ELISA and Western blot is the most widely used method for Lyme diagnosis. A two-tiered protocol is recommended by the Centers for Disease Control and Prevention (CDC): the sensitive ELISA test is performed first, and if it is positive or equivocal, then the more specific Western blot is run. The immune system takes some time to produce antibodies in quantity. After Lyme infection onset, antibodies of types IgM and IgG usually can first be detected respectively at 2-4 weeks and 4-6 weeks, and peak at 6-8 weeks. When an EM rash first appears, antibodies usually cannot yet be detected; therefore, antibody confirmation at that time has no diagnostic value and is not recommended. Up to 30 days after suspected Lyme infection onset, infection can be confirmed by detection of IgM or IgG antibodies; after that, it is recommended that only IgG antibodies be considered. A positive IgM and negative IgG test result suggests an early infection, especially if confirmed several weeks later by a positive IgG test result. The number of IgM antibodies usually collapses 4-6 months after infection, while IgG antibodies can remain detectable for years. After antibiotic treatment, antibody tests become less useful. People treated when they have an EM rash often subsequently test negative for Lyme antibodies, whether treatment was successful or instead Lyme goes on to cause further complications. People treated later usually test positive before and after treatment, regardless of treatment success or failure. Better diagnostic tests are needed.

The reliability of the CDC two-tiered protocol is controversial. Studies show the Western blot IgM has a specificity of 94-96% for people with clinical symptoms of early Lyme disease. The initial ELISA test has a sensitivity of about 70%, and in two-tiered testing, the overall sensitivity is only 64%, although this rises to 100% in the subset of people with disseminated symptoms, such as arthritis. Erroneous test results have been widely reported in both early and late stages of the disease, and can be caused by several factors, including antibody cross-reactions from other infections, including Epstein-Barr virus and cytomegalovirus, as well as herpes simplex virus. The overall rate of false positives is low, only about 1 to 3%, in comparison to a false-negative rate of up to 36% in the early stages of infection using two-tiered testing.

In Lyme carditis, electrocardiograms are used to evidence heart conduction abnormalities, while echocardiography may show myocardial dysfunction. Biopsy and confirmation of Borrelia cells in myocardial tissue may be used in specific cases but are usually not done because of risk of the procedure.

Polymerase chain reaction (PCR) tests for Lyme disease have also been developed to detect the genetic material (DNA) of the Lyme disease spirochete. Culture or PCR are the current means for detecting the presence of the organism, as serologic studies only test for antibodies of Borrelia. PCR has the advantage of being much faster than culture. However, PCR tests are susceptible to false positive results, e.g. by detection of debris of dead Borrelia cells or specimen contamination. Even when properly performed, PCR often shows false negative results because few Borrelia cells can be found in blood and cerebrospinal fluid (CSF) during infection.

Imaging

Neuroimaging is controversial in whether it provides specific patterns unique to neuroborreliosis, but may aid in differential diagnosis and in understanding the pathophysiology of the disease. Some evidence shows certain neuroimaging tests can provide data that are helpful in the diagnosis of a patient. Magnetic resonance imaging (MRI) and single-photon emission computed tomography (SPECT) are two of the tests that can identify abnormalities in the brain of a patient affected with this disease.

Treatment

Antibiotics are the primary treatment. The specific approach to their use is dependent on the individual affected and the stage of the disease. For most people with early localized infection, oral administration of doxycycline is widely recommended as the first choice, as it is effective against not only Borrelia bacteria but also a variety of other illnesses carried by ticks. People taking doxycycline should avoid sun exposure because of higher risk of sunburns. Doxycycline is contraindicated in children younger than eight years of age and women who are pregnant or breastfeeding; alternatives to doxycycline are amoxicillin, cefuroxime axetil, and azithromycin. Azithromycin is recommended only in case of intolerance to the other antibiotics. The standard treatment for cellulitis, cephalexin, is not useful for Lyme disease. When it is unclear if a rash is caused by Lyme or cellulitis, the IDSA recommends treatment with cefuroxime or amoxicillin/clavulanic acid, as these are effective against both infections. Individuals with early disseminated or late Lyme infection may have symptomatic cardiac disease, Lyme arthritis, or neurologic symptoms like facial palsy, radiculopathy, meningitis, or peripheral neuropathy. Intravenous administration of ceftriaxone is recommended as the first choice in these cases; cefotaxime and doxycycline are available as alternatives.

These treatment regimens last from one to four weeks. Neurologic complications of Lyme disease may be treated with doxycycline as it can be taken by mouth and has a lower cost, although in North America evidence of efficacy is only indirect. In case of failure, guidelines recommend retreatment with injectable ceftriaxone. Several months after treatment for Lyme arthritis, if joint swelling persists or returns, a second round of antibiotics may be considered; intravenous antibiotics are preferred for retreatment in case of poor response to oral antibiotics. Outside of that, a prolonged antibiotic regimen lasting more than 28 days is not recommended by IDSA, but there is also a different opinion by ILADS where treatment can be extended per need by patients. IgM and IgG antibody levels may be elevated for years even after successful treatment with antibiotics. As antibody levels are not indicative of treatment success, testing for them is not recommended.

Facial palsy may resolve without treatment, however, antibiotic treatment is recommended to stop other Lyme complications. Corticosteroids are not recommended when facial palsy is caused by Lyme disease. In those with facial palsy, frequent use of artificial tears while awake is recommended, along with ointment and a patch or taping the eye closed when sleeping. About a third of people with Lyme carditis need a temporary pacemaker until their heart conduction abnormality resolves, and 21% need to be hospitalized. Lyme carditis should not be treated with corticosteroids.

People with Lyme arthritis should limit their level of physical activity to avoid damaging affected joints, and in case of limping should use crutches. Pain associated with Lyme disease may be treated with nonsteroidal anti-inflammatory drugs (NSAIDs). Corticosteroid joint injections are not recommended for Lyme arthritis that is being treated with antibiotics. People with Lyme arthritis treated with intravenous antibiotics or two months of oral antibiotics who continue to have joint swelling two months after treatment and have negative PCR test for Borrelia DNA in the synovial fluid are said to have antibiotic-refractory Lyme arthritis; this is more common after infection by certain Borrelia strains in people with certain genetic and immunologic characteristics. Antibiotic-refractory Lyme arthritis may be symptomatically treated with NSAIDs, disease-modifying antirheumatic drugs (DMARDs), or arthroscopic synovectomy. Physical therapy is recommended for adults after resolution of Lyme arthritis. People receiving treatment should be advised that reinfection is possible and how to prevent it.

Current Diagnostic Limitations

Moreover, Lyme disease is a multisystem disease caused by B. burgdorferi. While serodiagnosis with high sensitivity for early disseminated and late arthritis stages of the disease is readily achieved, diagnosis of early Lyme disease and late persistent chronic stage of the disease remains suboptimal and challenging. During the growth from a log phase culture which primarily consists of spirochete form to stationary phase culture, B. burgdorferi develops multiple morphological variant forms, such as round bodies and aggregated biofilm-like microcolonies (MC). The current serodiagnostic tests for Lyme disease use antigens derived from mainly log phase growing cultures that largely consist of spirochetal form of the bacteria, and the significance of the morphological variant forms that are non-growing persisters developed under stress conditions with presumably different antigen expression in serodiagnosis of Lyme disease has remained largely unknown. As provided in the invention described herein, it was identified that only the microcolony persister (MC) form antigens (MPAs) but not antigens from the round body form could improve the sensitivity of serodiagnosis for Lyme disease, and that the MPAs combined with C6 peptide achieved an even higher sensitivity of serodiagnosis of Lyme disease.

The Centers for Disease Control and Prevention (CDC) recommends a two-tiered serologic test to detect the patient's antibody response to B. burgdorferi antigens (1, 6, 7). The first-tier consists of an enzyme-linked immunoassay (EIA) with whole cell lysates of B. burgdorferi, followed by a second-tier Western blot or a C6 peptide test (2). While the sensitivity of these tests is quite high for disseminated Lyme disease (>82%) (7), the sensitivity remains low for diagnosis of early stage Lyme disease with EM rash (30-40%) (3) and also for post-treatment Lyme disease syndrome (PTLDS) (50-60%) (3-7).

The main limitation of the current two-tier test is that it has limited sensitivity of detection for early Lyme and also PLTDS, but has worked well in early disseminated and especially Lyme arthritis where sensitivity could achieve over 90%. The current two tier test uses antigens mainly derived from growing Borrelia cultures. In contrast, the invention described herein provides significant advantages by providing a test that use antigens from non-growing Borrelia microcolony persister form, which has unique antigen expression that is different from the growing log phase Borrelia, and thus contributes to improved sensitivity of detection for both early Lyme when combined with C6 peptide and for PTLDS with or even without combination with C6.

Another disadvantage of the 2-tiered testing approach is that Western blot is too tedious. Recent studies indicate that C6 peptide, derived from VlsE protein when used as a stand-alone test is more sensitive than the current 2-tiered test for patients with early Lyme disease (64% vs. 48%) with comparable specificity (98.4% vs 99.5%) (8). Interestingly, when C6 test is added together with ELISA in a two-tiered ELISA format which is much simpler than ELISA plus Western blot and removes the complexity of the Western Blot, an increase in sensitivity in detecting early disease up to slightly over 50% sensitivity, with very good performance in Stage 2 and in Stage 3 illness 100% of the samples were observed (6-8). This indicates that it is possible to add other antigens together with C6 to improve the sensitivity of the current Lyme diagnosis. However, it remains a significant challenge to diagnose early Lyme disease as well as late stage PTLDS patients with the current two-tier test or even the new improved C6 plus whole cell lysate one tier test.

### Methods for Diagnosing Lyme Disease, in Particular Early Stage Lyme Disease, and Post-Treatment Lyme Disease Syndrome (PTLDS)

Included herein is a method of diagnosis Lyme disease, in particular early stage Lyme disease (e.g., within the first 4-6 weeks after tick bite, and is not early disseminated disease, and post-treatment Lyme disease syndrome (PTLDS)).

As described herein, methods for diagnosing Lyme disease in a subject are provided. In some examples, a sample (e.g., a bodily fluid comprising whole blood, a component of whole blood, plasma, serum, urine, cerebrospinal fluid, or synovial fluid) is obtained from the subject, the sample is then assayed for the level of antibodies to MPA antigens from Borrelia burgdorferi. In examples, the antigens comprise microcolony persister form antigens (MPA), and the subject is diagnosed with Lyme disease when detection of antibodies to MPA or the MPA in the sample is observed. For example, the subject is diagnosed with the Lyme disease if the level of the MPA is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in said test sample compared to a normal control.

In examples, the methods for diagnosing Lyme disease further comprises detecting C6 peptide comprising the sequence of MKKDDQIAAAIALRGMAKDGKFAVKDGE (SEQ ID NO: 1). For example, the detection of antibodies to the MPA in combination with detection of the C6 peptide provides for improved diagnosis.

The method of diagnosis is particularly beneficial for diagnosis of early Lyme disease. Early Lyme disease which is often characterized as within 4-6 weeks after the tick bite.

A subject diagnosed with Lyme disease according to the methods herein can be further administered a compound that is used to treat the Lyme disease. For example, the disclosed methods may be used for monitoring the effect of treatment by flowing the antibody levels to MPA.

Also provided herein are methods of detecting post-treatment Lyme disease syndrome (PTLDS) or Lyme disease in a subject. The methods comprise, for example, a sample (e.g., a bodily fluid comprising whole blood, a component of whole blood, plasma, serum, urine, cerebrospinal fluid, or synovial fluid) is obtained from the subject, and the presence of antigens from Borrelia burgdorferi are detected. In examples, the antigens comprise microcolony persister form antigens (MPA), and the detection of the MPA in the sample is indicative of PTLDS or Lyme disease.

In further examples, the method further comprises detecting the C6 peptide comprising MKKDDQIAAAIALRGMAKDGKFAVKDGE (SEQ ID NO: 1).

Also provided herein are methods for increasing the sensitivity of serodiagnosis for early Lyme disease and post-treatment Lyme disease syndrome in a subject. The method comprises, obtaining a sample from the subject and detecting the presence of antibodies to antigens from Borrelia burgdorferi, wherein the antigens comprise microcolony persister form antigens (MPA) and C6 peptide, and wherein detection of antibodies to the MPA and C6 or these antigens directly in the sample is indicative of PTLDS or Lyme disease and increases the sensitivity of serodiagnosis. In further examples, the method includes detecting the C6 peptide comprising MKKDDQIAAAIALRGMAKDGKFAVKDGE (SEQ ID NO: 1).

Also provided herein are methods of diagnosing Lyme disease in a subject, including obtaining a sample from the subject, and assaying the sample for i) the level of antibodies to antigens from Borrelia burgdorferi and/or ii) the level antigens from Borrelia burgdorferi, wherein the antigens comprise microcolony persister form antigens (MPA), and diagnosing the subject with Lyme disease when detection of the MPA in the sample is observed. In other examples, the sample may be assayed for the level of antibodies to antigens from Borrelia burgdorferi. In other examples, the sample is assayed for the level antigens from Borrelia burgdorferi.

### Methods for Monitoring the Effect of Treating Lyme Disease, in Particular Early Stage Lyme Disease, and Post-Treatment Lyme Disease Syndrome (PTLDS)

Included herein is a method of diagnosing or treating Lyme disease, in particular early stage Lyme disease, and post-treatment Lyme disease syndrome (PTLDS) in a subject in need thereof. In further embodiments, the method comprises administering to the subject an effective amount of the composition comprising an antibiotic.

In other embodiments, the methods for diagnosing and treating Lyme disease, in particular early stage Lyme disease, and post-treatment Lyme disease syndrome (PTLDS) comprise administering to a subject a composition comprising an antibiotic, in combination with methods for controlling the outset of symptoms. In particular, the combination treatment can include administering readily known treatments. Moreover, the described methods can be used to monitor the effect of Lyme disease, early Lyme disease or PTLDS treatment.

### Kits for Diagnosing and Treating Lyme Disease, in Particular Early Stage Lyme Disease, and Post-Treatment Lyme Disease Syndrome (PTLDS)

In aspects, a kit for diagnosing and treating Lyme disease, in particular early stage Lyme disease, and post-treatment Lyme disease syndrome (PTLDS) as well as all forms of Lyme disease are provided. In embodiments, the kit comprises the antigens derived from Borrelia microcolony persister form.

In embodiments, the kit is suitable for delivery (e.g., local injection) to a subject.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., Lyme disease, in particular early stage Lyme disease, and post-treatment Lyme disease syndrome (PTLDS)), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Morphological Variants of B. Burgdorferi in Different Growth Phases

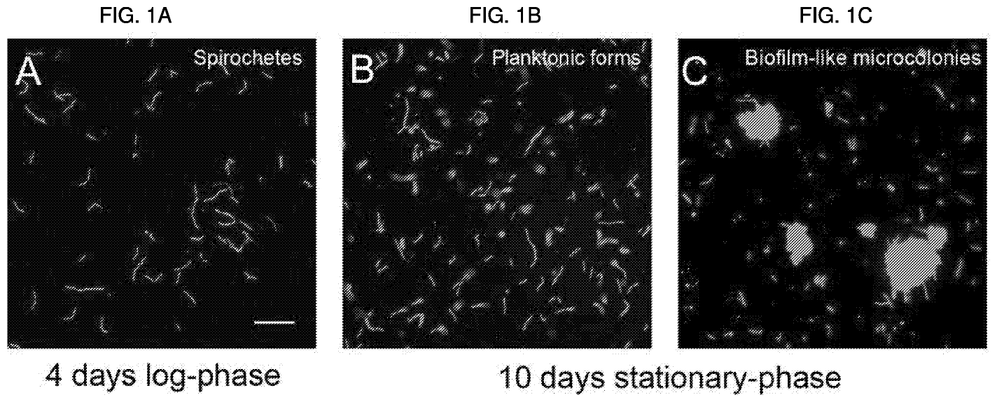

B. burgdorferi cultures at different time points of growth were detected by microscopy using SYBR Green I staining. As shown in FIGS. 1A-1C, B. burgdorferi bacteria in the 4-day old log phase culture were primarily in spirochetal form (LOG), whereas the 10-day old stationary phase culture contained in addition to spirochete form morphological variant forms including round body form and aggregated microcolony form (MC). Studies have demonstrated that the variant forms are persisters that are not sensitive to or killed by the current Lyme antibiotics (9, 10) and have unique protein expression differences (13) and ability to cause disease in mice with different forms (10).

To assess the utility of antigens prepared from different variant forms of B. burgdorferi for serodiagnosis of Lyme disease, each individual variant forms including microcolony (MC) form and planktonic form (SP) containing both spirochetes and round bodies from a 10-day old stationary phase culture, and spirochete form from a 4-day old log phase (LOG) culture were each individually isolated (FIGS. 1A-1C) and were subjected to protein extract preparations as described herein. The LOG, SP, and MC antigen preparations were evaluated for serodiagnosis of Lyme disease with patient samples as described below.

### Example 2: Antigens Derived from Biofilm-Like Microcolony (MC) Persisters Provided Better Sensitivity for Serodiagnosis of Lyme Disease Compared with Log Phase Spirochete Form (LOG) and Stationary Phase Planktonic Form (Spirochete Plus Round Bodies) (SP)

Using an initial panel of 65 Lyme disease serum samples from Columbia University Medical Center, the utility of the three different antigen preparations from LOG.Bb, SP.Bb, and MC.Bb, respectively, were first evaluated in diagnosis of Lyme disease by ELISA. Antigens were coated and incubated with serum samples from healthy controls, patients upon initial clinical presentation with EM skin lesion and patients with PTLDS or over 6 months of initial EM, followed by incubation with secondary antibody anti-IgM and anti-IgG. Using LOG.Bb as coating antigens, the total positive rate for patients was 55.38% (Table 1, below).

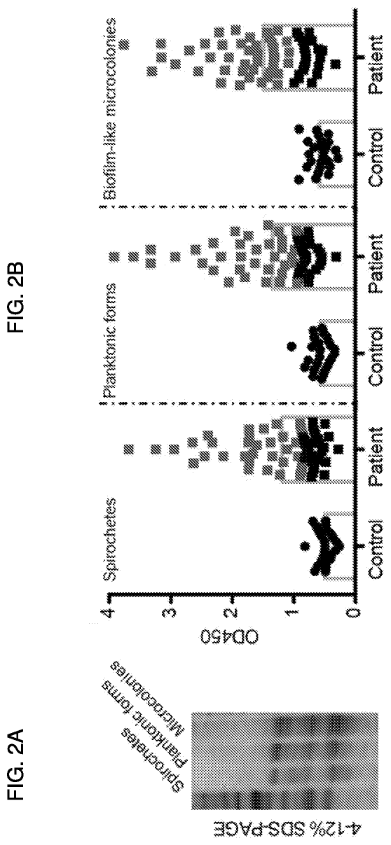

For SP.Bb lysate, the positive rate was the same (55.38%), which provides no significant difference in sensitivity compared with Log.Bb in serodiagnosis of Lyme disease. Interestingly, when MC.Bb lysate was used as coating antigens, the overall (total) positive rate increased considerably to 67.69%, showing a significant difference compared with Log.Bb or SP.Bb (Table 1 and FIG. 2B) (P=0.0037). Collectively, compared to LOG.Bb and SP.Bb, MC.Bb was the most effective antigen preparation as a first-tier ELISA for serodiagnosis of Lyme disease.

TABLE 1
Comparison of ELISA test using antigens derived from MC.Bb,
SP.Bb and LOG.Bb for diagnosis of Lyme disease*

| % | Pos | BL | Neg |
|---|---|---|---|
| LOG.Bb | 49.23 (32/65) | 6.15 (4/65) | 44.62 (29/65) |
| SP.Bb | 49.23 (32/65) | 6.15 (4/65) | 44.62 (29/65) |
| MC.Bb | 66.15 (43/65) | 1.54 (1/65) | 32.31 (21/65) |

*Pos, positive (>3SD from the mean of the controls); BL, borderline (between 2SD and 3SD from the mean of the healthy controls); Neg, negative (<2SD from the mean of the healthy controls). Sera responded to total antibody is the total number of samples contained IgM, IgG antibodies binding to the targets. P < 0.001 for Lyme disease versus normal healthy controls; P = 0.1869 for sera responding to stationary phase planktonic forms versus LOG Borrelia spirochete form. P = 0.0037 for sera responding to MC persister form versus LOG Borrelia spirochete form.

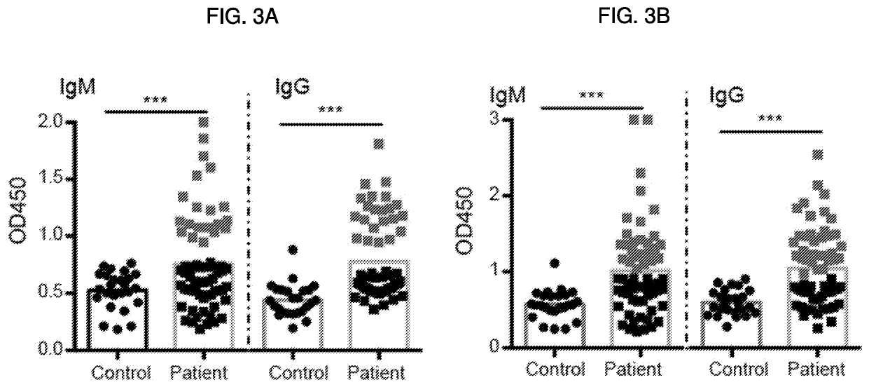

Next, the MC.Bb persister antigens (MPAs) were evaluated with patient serum samples in comparison with the current gold standard spirochete antigens (standard EIA). In this test, detection of both IgM and IgG were independently executed with the patient sera. Individual cutoffs were calculated for each antigen preparation by determining the mean absorbance of healthy control sera. As shown in Table 2 (below), the results indicated that the MPAs identified a higher number of positive patients than the standard EIA in diagnosing Lyme patients. Using log phase spirochete lysate as coating antigens, the total positive rate for Lyme disease subjects was 55.38% compared with healthy controls (FIG. 3A). However, using the MPAs, the total positive rate for Lyme disease increased to 69.23%, which showed a significant difference compared with the positive rate of 55.38% produced by log phase spirochete antigens (p<0.05) (FIG. 3B). The sensitivity of IgM response to LOG.Bb spirochete lysate was 30.77% but to MPA increased to 43.08%. In addition, IgG response to MPAs was significantly higher at 49.23% than log phase spirochete antigens at 33.85%. Since a recent study showed water induced round body form seemed to increase the sensitivity of serodiagnosis of Lyme disease (17), here the H₂O inducible round body form were produced as coating antigens, which produced the sensitivity of IgM and IgG as 32.3% and 40% respectively. Thus, compared with antigens from log phase spirochetes or H₂O inducible round body, MPAs produced the best positive rate of all these different antigen preparations.

The utility of the MPAs with C6 peptide was also compared. It was found that the MPAs alone (69.23%) provided a significantly better sensitivity than C6 peptide alone (47.69%) (p<0.001). Interestingly, the highest sensitivity of 72.31% positivity was achieved when the MPAs was combined with C6 in detecting Lyme disease (FIGS. 3A and 3B).

TABLE 2
Comparison of antigens derived from stationary phase biofilm-like MC persisters (MC.Bb) or water-inducible round body
(RB) form and log phase spirochete form (LOG.Bb) for serodiagnosis of Lyme disease using ELISA test (Columbia sample)*

| Antibody | LOG.Bb | | | MC.Bb | | | Water-inducible round body (RB) form | | |
|---|---|---|---|---|---|---|---|---|---|
| response | Pos | BL | Neg | Pos | BL | Neg | Pos | BL | Neg |
| IgM | 27.69(18/65) | 3.08(2/65) | 69.23(45/65) | 38.46(25/65) | 4.62 (3/65) | 56.92(37/65) | 26.15(17/65) | 6.15(4/65) | 67.69(44/65) |
| IgG | 33.85(22/65) | 0.00(0/65) | 66.15(43/65) | 44.62(29/65) | 4.62(3/65) | 50.77(33/65) | 33.85(22/65) | 6.15(4/65) | 4.62(39/65) |
| Total Aby | 52.31(34/65) | 3.08(2/65) | 44.62(29/65) | 66.15(43/65) | 4.62(2/65) | 30.77(20/65) | 50.77(33/65) | 4.62(3/65) | 44.62(29/65) |

*Pos, positive (>3SD from the mean of the controls); BL, borderline (between 2SD and 3SD from the mean of the healthy controls); Neg, negative (<2SD from the mean of the healthy controls). Sera responded to total antibody is the total number of samples containing either IgM, IgG or both antibodies binding to the target antigens. P < 0.001 for Lyme disease versus normal healthy controls; P = 0.0037 for sera responding to MC persister form versus LOG Borrelia spirochetes; P = 0.1729 for sera responding to H₂O inducible round body (RB) form versus LOG Borrelia spirochete form.

TABLE 3
Evaluation of antigen preparations derived from different variant forms using
well-characterized early Lyme and PTLDS serum samples from SLICE studies*

| | Sample type | | | | | | | | |
|---|---|---|---|---|---|---|---|---|---|
| | LOG.Bb | | | MC.Bb | | | C6 peptide | | |
| | Pos | BL | Neg | Pos | BL | Neg | Pos | BL | Neg |
| Early | | | | | | | | | |
| Lyme | | | | | | | | | |
| IgM | 35.00(21/60) | 8.33(5/60) | 56.67(34/60) | 43.33(26/60) | 3.33(2/60) | 53.33(32/60) | 48.33(29/60) | 1.67(1/60) | 50.00(30/60) |
| IgG | 21.67(13/60) | 10.00(6/60) | 68.33(41/60) | 26.67(16/60) | 6.67(4/60) | 66.67(40/60) | 36.67(22/60) | 5.00(3/60) | 58.33(35/60) |
| Total | 41.67(25/60) | 8.33(5/60) | 50.00(30/60) | 48.33(29/60) | 6.67(4/60) | 45.00(27/60) | 50.00(30/60) | 1.67(1/60) | 48.33(29/60) |
| Aby | | | | | | | | | |
| PTLDS | | | | | | | | | |
| IgM | 6.67(4/60) | 6.67(4/60) | 86.67(52/60) | 11.67(7/60) | 1.67(1/60) | 86.67(52/60) | 16.67(10/60) | 3.33(2/60) | 80.00(48/60) |
| IgG | 36.67(22/60) | 1.67(1/60) | 61.67(37/60) | 46.67(28/60) | 8.33(5/60) | 45.00(27/60) | 38.33(23/60) | 3.33(2/60) | 58.33(35/60) |
| Total | 38.33(23/60) | 3.33(2/60) | 58.33(35/60) | 51.67(31/60) | 8.33(5/60) | 40.00(24/60) | 45.00(27/60) | 0.00(0/60) | 55.00(33/60) |
| antibody | | | | | | | | | |

*Pos, positive (>3SD from the mean of healthy controls); BL, borderline (between 2SD and 3SD from the mean of healthy controls); Neg, negative (<2SD from the mean of healthy controls). Sera responding to total antibody is the total number of samples containing either IgM, IgG or both antibodies binding to the target antigens. P = 0.07375 for early Lyme disease sera responding to MC persister form versus LOG Borrelia spirochete form; P = 0.00053 for PTLDS sera responding to MC persister form versus LOG Borrelia spirochete.

### Example 3. Antigens Derived from Biofilm-Like Microcolony (MC) Persisters Express Unique Proteins Compared with Log Phase Spirochete Form (LOG) and Stationary Phase Planktonic Form (Spirochete Plus Round Bodies) (SP)

Sodium dodecyl sulfate polyacrylamide gel electrophoresis (SDS-PAGE) was performed by standard methods. The lysates from different log phase, microcolonies, and stationary phase planktonic B. burgdorferi N40 strain were boiled in sample buffer for 10 min and run on 12.5% SDS-PAGE gels at 20 mA/gel at room temperature. The proteins were transferred to PVDF membrane using semi-dry transfer at 15 V for 2 h. After transfer, the membrane was blocked with 3% non-fat milk diluted in IX PBST (0.01 mol/L, pH 7.0, PBS with 0.05% Tween 20) for 1 h at room temperature. The membrane was washed with 1× PBST and membrane strips were incubated at 4° C. with patient sera diluted 1:100 with 1× PBST containing 3% non-fat dried milk for overnight, washed three times with 1× PBST for 5 min each time and then incubated for 1 h with goat anti-human IgG/A/M horseradish peroxidase-conjugated secondary antibodies (Invitrogen A18847) (1:5000 dilution), respectively. After washing six times with 1× PBST for 5 mm per wash, ECL prime was used for detection and the blots were incubated with the detection solution for 5 min film development following the manufacturer's instruction.

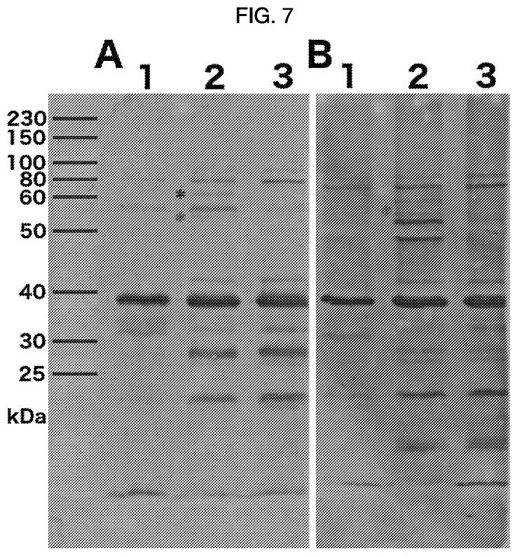

As depicted in the image of the blot in FIG. 7, patient A (left panel) and patient B (right panel) had antibodies that reacted with unique protein bands (marked with colored asterisks) from microcolony cell lysate as antigen but not from antigens from Log phase spirochetes or stationary phase planktonic form.

### Example 4: Evaluation of Antigen Preparations Derived from Different Variant Forms Using Well-Characterized Early Lyme and PTLDS Serum Samples from the Johns Hopkins Slice Study

Next, a larger number of 140 (60 early Lyme, 60 PTLDS, 20 healthy control) blind-coded serum samples were used to validate the results above. All of the samples had been tested by the CDC two-tier test using with commercial kits: ELISA was performed with Lyme Screen II (bioMerieux), and IgM and IgG Western blot was performed with Marblot (MarDx), and the results of clinical laboratory diagnosis for the 120 Lyme patients (60 early Lyme+60 PTLDS) are shown in Table 4, below.

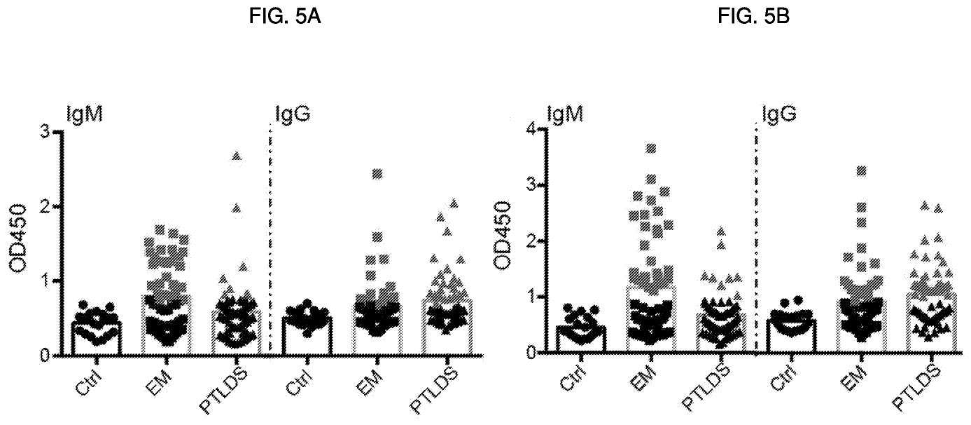

Based on the study above with the 65 serum samples, antigens prepared from SP (stationary phase planktonic forms—spirochete and round bodies) or water-inducible round body form did not offer any advantage to improve the sensitivity of the test, they were not included in the subsequent analyses with the 140 serum samples. Instead, antigens were used from log phase spirochetes (LOG.Bb), MPA, and C6 peptide in ELISA test with the 140 serum samples from 60 early Lyme and 60 PTLD and 20 health controls. The results are presented in FIGS. 5A and 5B and Table 5. For early Lyme, IgM response was positively detected in 43.33% using spirochete antigens, while IgG response was positively detected in 31.67%. However, using MPA, the IgM and IgG positive rates for early Lyme disease were 46.67% and 33.33%, respectively (Table 5).

Next the diagnostic potential of MPA for the detection of antibodies in patients with PTLDS was tested. It was found that combined IgM and IgG response to MPA was positively detected in 60.00% of the PTLDS patients compared with 41.67% with LOG spirochete antigens. Furthermore, IgG response to persister forms was significantly higher at 55.00% than IgM response at 13.33%. Taken together, the MPA (microcolony persister antigens) described herein were much more effective for diagnosis of Lyme disease, especially for PTLDS.

TABLE 4
Comparison of results of MC.Bb and LOG.Bb with clinical laboratory diagnosis
using ELISA and Western blot for the 120 serum samples from the SLICE study

| | Sample type | | | | | | | | | |
|---|---|---|---|---|---|---|---|---|---|---|
| | Lyme-Western Blot | | Lyme-ELISA | | LOG.Bb | | MC.Bb | | C6 peptide | |
| | Pos | Neg | Pos | Neg | Pos | Neg | Pos | Neg | Pos | Neg |
| Early | 40.00 | 60.00 | 51.67 | 48.33 | 50.00 | 50.00 | 55.00 | 45.00 | 51.67 | 48.33 |
| Lyme | (24/60) | (36/60) | (31/60) | (29/60) | (30/60) | (30/60) | (33/60) | (27/60) | (31/60) | (29/60) |
| PTLDS | 26.67 | 73.33 | 46.67 | 53.33 | 41.67 | 58.33 | 60.00 | 40.00 | 45.00 | 55.00 |
| | (16/60) | (54/60) | (28/60) | (32/60) | (25/60) | (35/60) | (36/60) | (24/60) | (27/60) | (33/60) |

TABLE 5
Individual IgM and IgG response to Log.Bb and
MC.Bb (MPA) in 120 Lyme serum samples (60 early
Lyme and 60 PTLDS) in the JHU SLICE study

| | IgM | IgG | IgG + IgM |
|---|---|---|---|
| EIA | | | |
| Positive hits | 28.33%(34/120) | 35.00%(42/120) | 45.83%(55/120) |
| Early Lyme | 43.33%(26/60) | 31.67%(19/60) | 50.00%(30/60) |
| (60) | | | |
| PTLDS (60) | 13.33%(8/60) | 38.33%(23/60) | 41.67%(25/60) |
| PA | | | |
| Positive hits | 30.00%(36/120) | 44.17%(53/120) | 57.50%(69/120) |
| Early Lyme | 46.67%(28/60) | 33.33%(20/60) | 55.00%(33/60) |
| (60) | | | |
| PTLDS(60) | 13.33%(8/60) | 55.00%(33/60) | 60.00%(36/60) |
| p value | | | |
| Early Lyme | 0.130020823 | 0.259831964 | 0.073750341 |
| PTLDS | 0.641591574 | 0.001376927 | 0.000533096 |
| Total | 0.101160523 | 0.002390014 | 0.000231249 |

### Example 5: Combination of MPA and C6 Peptide Improves Sensitivity of Serodiagnosis for Both Early Lyme and PTLD Patients

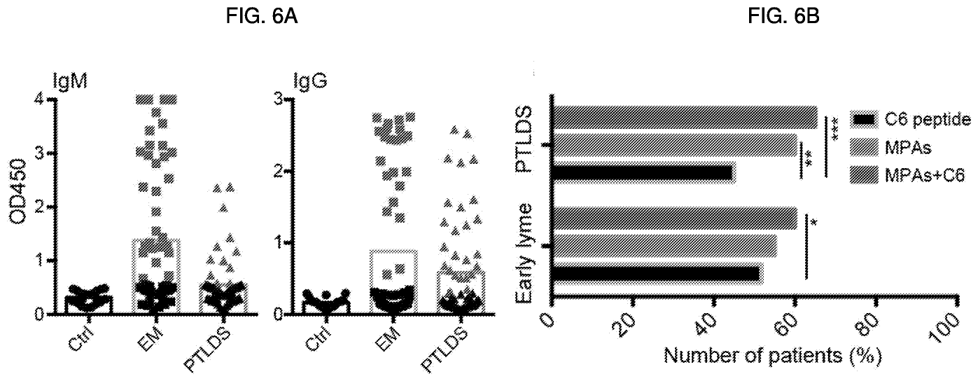

The utility of MPA with the commercial C6 peptide ELISA was compared for serodiagnosis of Lyme disease using the well-characterized 140 blind coded serum samples. The results are presented in Table 6 below. The total positive rate reactive with C6 peptide for the early Lyme disease patient samples was 51.67% ( 31/60). However, for the PTLDS patient sera, C6 peptide positive response was only 45.00% ( 27/60). More importantly, MPA alone (57.5%) were significantly more sensitive than C6 alone (45%) (p<0.05%). In combination analysis, the MC persister antigens (MPAs)+C6 yielded the highest sensitivity at 62.5% ( 75/120) positivity in detecting overall Lyme disease (FIGS. 6A and 6B) (Table 6). The MPA+C6 gave the best sensitivity for detecting both early Lyme (60%) (p<0.05) and PTLDS (65%) (p<0.05) compared with the current C6 peptide at 51.67% and 45% respectively (Table 6).

Taken together, these data demonstrated that MC persister antigens (MPA) plus C6 peptide improved the sensitivity of the current serodiagnosis of Lyme disease for both early Lyme and PTLDS patients.

TABLE 6
Persister antigens (MC.Bb or MPA) in combination with C6
peptide improved the sensitivity of serodiagnosis using
well-characterized early Lyme and PTLDS serum samples

| | C6 peptide | PA | PA + C6 |
|---|---|---|---|
| Total positive | 48.33%(58/120) | 57.50%(69/120) | 62.50%(75/120) |
| hits | | | |
| Early Lyme | 51.67%(31/60) | 55.00%(33/60) | 60.00%(36/60) |
| (60) | | | |
| PTLDS(60) | 45.00%(27/60) | 60.00%(36/60) | 65.00%(39/60) |
| p value | C6 vs PA | | C6 vs C6 + Ps |
| Early Lyme | 0.108786322 | | 0.02625013 |
| PTLDS | 0.002338313 | | 0.000188988 |
| Total | 0.001145022 | | 2.73846E−05 |

### Discussion

Although serodiagnosis of early disseminated and late arthritis stages of the disease is readily achieved with high sensitivity with the current CDC two-tier test (6, 7, 19), diagnosis of early Lyme disease and late persistent form of the disease has remained challenging. The current first-tier EIA tests use antigens derived from mainly log phase cultures that largely consist of spirochete form of the bacteria. As described herein, inclusion of antigens from Borrelia persisters such as biofilm-like microcolonies improved the sensitivity of diagnosis of Lyme disease. Indeed, it was found that the antigens isolated from biofilm-like MC persisters (MPAs) significantly improved the sensitivity of diagnosis of PTLDS patients compared to antigens derived from log phase spirochetes or C6 peptide (p<0.05), and in particular, combination of MPA with C6 peptide further enhanced the sensitivity of diagnosis for both early Lyme and PTLDS patients (p<0.05).

The current Lyme diagnostic test EIA uses antigens prepared from mostly log phase cultures and high in vitro passaged cultures (20), which may lack some antigens that are expressed in vivo and are present in the MPA. Meanwhile, B. burgdorferi was shown to have elevated expression of virulence related proteins in biofilm-like microcolonies compared to spirochetes using proteomic analysis (13), a finding that supports that the microcolonies produced antigens that more mimic those expressed in vivo and their inclusion improves serodiagnosis of Lyme disease.

Although antigens from biofilm-like MC persisters improve the sensitivity of the current serodiagnostic tests, it is worth noting that antigens from SP prepared from the 10 day old stationary phase planktonic form (both spirochete and round body) did not improve the sensitivity of the test. This indicated that not all antigens expressed from stationary phase B. burgdorferi cells are useful for improving the sensitivity of serodiagnosis of Lyme disease, but only the aggregated biofilm-like microcolonies express unique antigens that are useful for improving the sensitivity of the ELISA test.

A recent study used antigens derived from a 2 hr water-inducible round body form of B. burgdorferi for serodiagnosis of Lyme disease and claimed higher sensitivity than the standard EIA (17). However, in this study, no antigens were found from 2 hr water-induced round body form that could improve the sensitivity of serodiagnosis of Lyme disease. Instead, a different method was used to prepare the persister antigens derived from aggregated biofilm-like microcolonies isolated from 10 day old stationary phase cultures that are enriched in naturally formed persisters rather than those from a short 2 hr artificial water treatment which would have a different antigen expression, and it was found that MPAs offered significantly better sensitivity than the current tests for serodiagnosis of Lyme disease.

Although it was found that MPA produced improved sensitivity of serodiagnosis of Lyme disease, the specific antigens in the MPA responsible for the improved sensitivity remain to be identified. Future studies are determined to identify which antigens are responsible for the improved sensitivity conferred by the MPA. Nevertheless, just like the current serodiagnostic EIA tests that use crude lysates containing mixed antigens of mostly log phase cultures, the mixed nature of MPA derived from microcolonies should not affect its use for improved serodiagnosis of Lyme disease. However, proper quality control of possible batch to batch variations in MPA preparation is needed. The study herein highlights the importance of the inclusion of persister antigens for improved diagnosis of Lyme disease and may have implications for diagnosis of other microbial infections (bacterial, fungal or parasitic) by inclusion of antigens derived from their respective persister forms.

The observation that more PTLDS patients reacted with the MPA than with the log phase spirochete antigens is of interest. This may suggest that the PTLDS patients may be preferentially infected with or harbor biofilm-like microcolony persisters either from the very beginning of the tick bite or from prolonged persistent infection (10) such that they are likely to develop more antibodies to microcolony persisters than to log phase growing spirochetes. It would be of interest to determine if positive antibody response to the MPA could serve as a biomarker for PTLDS and treatment response and if MPA-positive patients tend to develop more persistent or severe disease or PTLDS or are associated with poor treatment response or poor outcome, or conversely, decrease in antibody levels to MPA correlates with treatment effect.

The quality of the serum samples used is vital for evaluation of the serodiagnostic tests of Lyme disease. Inclusion of seronegative samples from clinically diagnosed early Lyme and PTLDS patients is critical for evaluation of new tests that could potentially improve the sensitivity of the current existing test. This is because the current two-tier test already has a very good sensitivity in detecting early disseminated disease and late stage Lyme arthritis (19), and any new test will not likely improve upon near perfect test for this subgroup of patients (19). For example, a panel of 100 well-curated CDC two-tier test positive Lyme patient serum samples were evaluated from Boston Children's Hospital and found that the MPA test was as good as the current two-tier test in detecting the 100 seropositive samples (Lise Nigrovic). Thus, the CDC two-tier test positive samples while useful for self-validation are not useful for evaluating new test that could improve the sensitivity of the current test.

The study evaluated close to 200 samples from Lyme disease patients in two batches of samples from Columbia University (65 Lyme samples) and Johns Hopkins (120 samples) and already represents one of the largest number of samples for studies that evaluated serodiagnostic tests of Lyme disease. Thus, the findings described herein are robust and reliable.

Further studies on larger number of samples in comparison with other commercial tests are performed to validate the utility of the MPAs for improved diagnosis of Lyme disease in the future. Moreover, the MPA test is adapted to a lateral flow immunoassay (LFI) that can produce results in 10-15 minutes for more rapid and convenient point-of-care (POC) application in doctor's office or mass screening.

### Materials and Methods

### Bacterial Strain and Culture.

Borrelia burgdorferi B31 strain (ATCC 35210) was obtained from American Type Tissue Collection (ATCC). B. burgdorferi was cultured in BSK-H medium (HiMedia Laboratories Pvt. Ltd.) supplemented with 6% rabbit serum (Sigma-Aldrich) as described previously (15). The culture medium was filter-sterilized by 0.2 μm filter. Cultures were prepared by inoculation of B. burgdorferi B31 at 1:100 dilution and incubated in sterile 50 ml conical tubes (BD Biosciences, California, USA) at 33° C. without shaking with a humidified atmosphere of 5% CO2. A 4-day old culture was used as log phase culture which consisted of growing spirochetal form, and a 10-day old culture was used as stationary phase B. burgdorferi culture which contained morphological variant forms including spirochete, round body, microcolony forms (9).

### Isolation of Variant Forms and Antigen Preparation

B. burgdorferi is known to develop from the spirochete form to morphological variant forms such as round bodies and biofilm-like microcolonies as the log phase culture grows into old stationary phase culture (9). To isolate B. burgdorferi microcolony form, 10-day old stationary phase cultures were harvested using a low speed at 800 g for 15 min. The supernatant and the cell pellet were collected as the planktonic and biofilm-like microcolony forms, respectively. The morphology of the variant forms was confirmed by SYBR Green/PI staining followed by microscopy as described previously (16). A 4-day old B. burgdorferi was collected as the log phase culture containing spirochetal form (15). In addition, water-inducible round body form of B. burgdorferi was prepared by treatment of log phase culture with H₂O for two hours as described previously (17). All the different forms of B. burgdorferi were re-suspended in 300 μl lysis buffer (PBS containing 2% SDS, 1 mM EDTA and protease inhibitor) followed by sonication to prepare whole cell lysates. C6 peptide (MKKDDQIAAAIALRGMAKDGKFAVKDGE (SEQ ID NO: 1)), the main invariant epitope from the variable protein VlsE (18), was synthesized and used as a positive control antigen in serodiagnosis in this study.

### Patient Serum Samples

De-identified human serum samples were obtained from two different sources. One source was from Columbia University Medical Center, which had 65 serum samples from patients diagnosed with Lyme disease with an erythema migrans (EM) rash, or from patients with diagnosis of PTLDS with persisting symptoms over 6 months despite standard treatment. In addition, 25 healthy individual samples were collected as negative controls. The other serum sample collection was from the SLICE studies at Johns Hopkins Lyme Disease Center, consisting of 140 specimens, where 60 were from patients in the acute phase of Lyme disease with EM and 60 were PTLD patients, and 20 healthy controls.

### ELISA

Antigens were diluted in 100 mM carbonate-bicarbonate buffer, pH 9.6, and applied to Immobilizer Amino microtiter plates (Nunc, Inc). Two different forms of B. burgdorferi lysate were applied at 100 ng per well and the C6 peptide was applied at 500 ng per well. The plates were blocked using 2% bovine serum albumin (BSA) in phosphate buffered saline (PBS) with 0.01% Tween 20 (PBST), and plates were washed extensively with PBST for all steps. Serum samples were diluted in 1:100 in 2% BSA in PBST. Secondary antibodies against human IgG and IgM conjugated to horseradish peroxidase were used at 1:2000 and 1:4000 dilutions, respectively. All steps were carried out either for 1 h at room temperature or overnight at 4° C. ELISA results were quantified using tetramethylbenzidine (TMB) substrate according to the manufacturer's instructions (Life Technologies). The enzyme reaction was then terminated with 1% sodium dodecyl sulfate solution. The optical density (OD) was measured at 450 nm with an ELISA microplate reader (Bio-Tek). The cut-off OD value was defined as the mean OD plus 3 standard deviations (SDs) for 25 healthy control serum samples.

### Statistical Analysis

Statistical analyses were performed using Prism 6.0 (Graph Pad, La Jolla, Calif.). Statistical differences in the mean absorbance of IgM and IgG binding to proteins were compared using a Kruskal-Wallis nonparametric test, followed by a Dunn multiple comparison test. The diagnostic performance of each antigen preparations was compared pair-wise using the area under the curve (AUC) from receiver operator characteristic (ROC) analysis. The statistical analysis of differences of positive rate between two test antigen preparations was compared by a Chi-square test for paired data.

### REFERENCES

1. CDC. Lyme Disease. 2017 [Jun. 13, 2018]. Available from: http://www.cdc.gov/lyme/.
2. Radolf J D, Caimano M J, Stevenson B, Hu L T. Of ticks, mice and men: understanding the dual-host lifestyle of Lyme disease spirochaetes. Nat Rev Microbiol. 2012; 10(2):87-99.
3. Steere A C, Strle F, Wormser G P, Hu L T, Branda J A, Hovius J W, et al. Lyme borreliosis. Nature reviews Disease primers. 2016; 2:16090.
4. Wormser G P, Dattwyler R J, Shapiro E D, Halperin J J, Steere A C, Klempner M S, et al. The clinical assessment, treatment, and prevention of lyme disease, human granulocytic anaplasmosis, and babesiosis: clinical practice guidelines by the Infectious Diseases Society of America. Clinical infectious diseases: an official publication of the Infectious Diseases Society of America. 2006; 43(9):1089-134.
5. CDC. Post-Treatment Lyme Disease Syndrome. 2017 [Jul. 3, 2018]. Available from: http://www.cdc.gov/lyme/postLDS/index.html.
6. Molins C R, Delorey M J, Sexton C, Schriefer M E. Lyme Borreliosis Serology: Performance of Several Commonly Used Laboratory Diagnostic Tests and a Large Resource Panel of Well-Characterized Patient Samples. J Clin Microbiol. 2016; 54(11):2726-34.
7. Moore A, Nelson C, Molins C, Mead P, Schriefer M. Current Guidelines, Common Clinical Pitfalls, and Future Directions for Laboratory Diagnosis of Lyme Disease, United States. Emerg Infect Dis. 2016; 22(7).
8. Branda J A, Strle K, Nigrovic L E, Lantos P M, Lepore T J, Damle N S, et al. Evaluation of Modified 2-Tiered Serodiagnostic Testing Algorithms for Early Lyme Disease. Clinical infectious diseases: an official publication of the Infectious Diseases Society of America. 2017; 64(8): 1074-80.
9. Feng J, Auwaerter P G, Zhang Y. Drug Combinations against Borrelia burgdorferi Persisters In Vitro: Eradication Achieved by Using Daptomycin, Cefoperazone and Doxycycline. PLoS One. 2015; 10(3):e0117207.
10. Feng J, Li T, Yee R, Yuan Y, Bai C, Cai M, et al. Stationary phase persister/biofilm microcolony of Borrelia burgdorferi causes more severe disease in a mouse model of Lyme arthritis: implications for understanding persistence, Post-treatment Lyme Disease Syndrome (PTLDS), and treatment failure. Discov Med. 2019; 27(148):125-38.
11. Merilainen L, Brander H, Herranen A, Schwarzbach A, Gilbert L. Pleomorphic forms of Borrelia burgdorferi induce distinct immune responses. Microbes and infection. 2016; 18(7-8):484-95.
12. Feng J, Shi W, Zhang S, Zhang Y. Persister mechanisms in Borrelia burgdorferi: implications for improved intervention. Emerg Microbes Infect. 2015; 4(8):e51.
13. Feng J, Zhang Y. Proteomic Analyses of Morphological Variants of Borrelia burgdorferi Shed New Light on Persistence Mechanisms: Implications for Pathogenesis, Diagnosis and Treatment. https://wwwbiorxivorg/content/101101/501080v1. 2018.
14. Zhang Y. Persisters, Persistent Infections and the Yin-Yang Model. Emerg Microb Infect (Nature Publishing Group). 2014; 3, e3; doi:10.1038/emi.2014.3.
15. Feng J, Wang T, Shi W, Zhang S, Sullivan D, Auwaerter P G, et al. Identification of Novel Activity against Borrelia burgdorferi Persisters Using an FDA Approved Drug Library. Emerg Microb Infect. 2014; Jul. 2, 2014:3, e49; doi:10.1038/emi.2014.53.
16. Feng J, Wang T, Zhang S, Shi W, Zhang Y An optimized SYBR Green I/PI assay for rapid viability assessment and antibiotic susceptibility testing for Borrelia burgdorferi. PLoS One. 2014; 9(11):e111809.
17. Garg K, Merilainen L, Franz O, Pirttinen H, Quevedo-Diaz M, Croucher S, et al. Evaluating polymicrobial immune responses in patients suffering from tick-borne diseases. Scientific reports. 2018; 8(1):15932.
18. Liang F T, Aberer E, Cinco M, Gern L, Hu C M, Lobet Y N, et al. Antigenic conservation of an immunodominant invariable region of the VlsE lipoprotein among European pathogenic genospecies of Borrelia burgdorferi SL. J Infect Dis. 2000; 182(5):1455-62.
19. Aguero-Rosenfeld M E, Wang G, Schwartz I, Wormser G P. Diagnosis of lyme borreliosis. Clin Microbiol Rev. 2005; 18(3):484-509.
20. Engstrom S M, Shoop E, Johnson R C. Immunoblot interpretation criteria for serodiagnosis of early Lyme disease. J Clin Microbiol. 1995; 33(2):419-27.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A method of diagnosing Lyme disease in a subject, comprising:
obtaining a sample from the subject,
assaying the sample for i) the level of antibodies to antigens from Borrelia burgdorferi and/or ii) the level of antigens from Borrelia burgdorferi, wherein the antigens comprise microcolony persister form antigens (MPA), and
diagnosing the subject with Lyme disease when detection of antibodies to MPA or the MPA in the sample is observed.

2. The method of claim 1 wherein the sample is assayed for the level of antibodies to antigens from Borrelia burgdorferi.

3. The method of claim 1 wherein the sample is assayed for the level antigens from Borrelia burgdorferi.

4. The method of claim 1 wherein said subject is diagnosed with the Lyme disease if the level of the antibody is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in said test sample compared to a normal control.

5. The method of claim 1 further comprising detecting antibody to C6 peptide comprising MKKDDQIAAAIALRGMAKDGKFAVKDGE (SEQ ID NO: 1) in combination with detecting the antibody to MPA.

6. The method of claim 1 wherein the Lyme disease comprises early Lyme disease.

7. The method of claim 1 wherein the Lyme disease comprises Post-treatment Lyme disease syndrome or persistent or chronic Lyme disease.

8. The method of claim 1 wherein the sample comprises a bodily fluid from the subject.

9. The method of claim 8 wherein the bodily fluid comprises whole blood, a component of whole blood, plasma, serum, urine, cerebrospinal fluid, or synovial fluid.

10. The method of claim 1 wherein said assaying comprises an enzyme-linked immunosorbent assay (ELISA), an antigen capture assay, flow cytometry, immunoblot, lateral flow assay (LFA), a Western blot, a mass spectrometry assay, immunoprecipitation, immunodiffusion, quantum dot, immunocytochemistry, radioimmunoassay, or any combination thereof.

11. A method of detecting post-treatment Lyme disease syndrome (PTLDS) or Lyme disease in a subject, comprising:
obtaining a sample from the subject,
detecting in the sample i) the level of antibodies to antigens from Borrelia burgdorferi and/or ii) the level antigens from Borrelia burgdorferi, wherein the antigens comprise microcolony persister form antigens (MPAs),
wherein detection of the antibodies or antigens in the sample is indicative of PTLDS or Lyme disease.

12. The method of claim 11 wherein the level of antibodies to antigens from Borrelia burgdorferi is detected.

13. The method of claim 11 wherein the level of antigens from Borrelia burgdorferi is detected.

14. The method of claim 1 further comprising detecting antibody to C6 peptide comprising MKKDDQIAAAIALRGMAKDGKFAVKDGE (SEQ ID NO: 1) in combination with detecting the antibody to MPA.

15-17. (canceled)

18. The method of claim 1 further comprising detecting C6 peptide comprising MKKDDQIAAAIALRGMAKDGKFAVKDGE (SEQ ID NO: 1) in combination with detecting the antibody or antigens.

19. A kit comprising
(a) at least two agents selected from the group consisting of (i) microcolony persister form antigens (MPA); and (ii) C6 peptide, and
(b) instructions for using the agent for diagnosing a Lyme disease or post-treatment Lyme disease syndrome (PTLDS), for identifying whether a subject is at risk of developing the Lyme disease or PTLDS, for determining the prognosis of the Lyme disease or PTLDS, for determining the progression of Lyme disease or PTLDS, for assessing the efficacy of a treatment for the Lyme disease or PTLDS, and/or for adjusting the dose of a compound during the treatment of Lyme disease or PTLDS, or
A diagnostic system comprising
(a) an assortment, collection, or compilation of test results representing the level of antibodies to antigens from Borrelia burgdorferi, wherein the antigens comprise microcolony persister antigens (MPA) or C6 peptide in a plurality of test samples;
(b) a means for computing an index value using said level, wherein the index value comprises a diagnostic, prognostic, progression, or treatment response; and
(c) a means for reporting the index value.

20. (canceled)

21. A method for determining the efficacy of a therapeutic regimen in a subject having Lyme disease, the method comprising:
a) detecting the presence of (i) antibodies to antigens from Borrelia burgdorferi or (ii) antigens from Borrelia burgdorferi, wherein the antigens comprise microcolony persister form antigens (MPA), in the subject prior to the initiation of treatment;
b) detecting the presence of (i) antibodies to antigens from Borrelia burgdorferi or (ii) antigens from Borrelia burgdorferi, wherein the antigens comprise microcolony persister form antigens (MPA) in the subject after initiation of the therapeutic regimen; and
wherein a decrease in the antibodies or antigens is a positive indicator of the efficacy of the therapeutic regimen.

22-27. (canceled)


SEQUENCE LISTING (1 sequence)

SEQ ID NO: 1 - protein, 28 aa, Borrelia burgdorferi
MKKDDQIAAAIALRGMAKDGKFAVKDGE